(19) United States
(12) Patent Application Publication
(10) Pub. No.: US 2014/0018902 A1
(43) Pub. Date: Jan. 16, 2014

(54) TAILOR-MADE STENT GRAFT AND PROCEDURE FOR MINIMALLY INVASIVE ANEURYSM REPAIR WITH NOVEL TAILOR-MADE BALLOON, NOVEL GUIDEWIRE, AND NOVEL CAPSULATED BIOGLUE

(71) Applicant: David MYR, Jerusalem (IL)

(72) Inventor: David MYR, Jerusalem (IL)

(73) Assignee: Makor Issues and Rights Ltd., Jerusalem (IL)

(21) Appl. No.: 13/547,467

(22) Filed: Jul. 12, 2012

(51) Int. Cl. A61F 2/06

(52) U.S. Cl. 623/1.13

(57) ABSTRACT

An individually tailored endovascular stent graft device and procedure is for performing a no-cut repair of different aneurysm types: ascending, descending, arch, abdominal and cerebral aneurysms. Many aneurysm types can thus be treated without the need for open heart surgery. The stent may be biomaterial based (collagen-based in the preferred embodiment). No shape-memory metals need be used therefore allowing better implantation flexibility and better patient recovery. The stent may be fixated in the designated treatment area by remotely activating individually capsulated bicomponent biological glue by UV light/ultrasound means, wherein each component of the glue is coated separately. Further presented is a method for calculating stent graft implantation path by determining the sequence of implantation points through computer simulation means, wherein the implantation is done by discrete pulses.

### FIELD OF THE INVENTION

The present invention relates to biotechnology field, and more specifically to devices and methods for performing minimally invasive endovascular procedures. More specifically, the device of the present invention can be used for treating aorta and cerebral aneurysms.

### OBJECTS OF THE INVENTION

The present invention aims to overcome the shortcomings in the state of the art.

It is a main object of the invention to provide an individually tailored device, a method and a procedure for treating different types of aneurysms by minimally invasive procedure.

It is therefore a main object of the invention to improve the overall health of patient's cardiovascular system by implanting stent graft in places of aneurysm/s.

It is a further object of the invention to disclose a method and a procedure where biological glue can be remotely released and activated only at treatment site.

It is a further object of the present invention to disclose stent graft for ascending aorta aneurysm repair, wherein such stent graft is produced without shape memory metal, has the flexibility to pass the aorta arch and better patient recovery chances.

### DETAILED DESCRIPTION OF THE INVENTION

### Summary of the Invention

In order to accomplish the aforementioned and other objects of the invention and to overcome the shortcomings of the prior art, this invention provides an individually tailored endovascular stent graft device, method and procedure for performing a no-cut repair procedure of ascending, descending, arch, abdominal aorta aneurysms, as well as the cerebral aneurysm. The stent will be fixated in the designated treatment area by using remotely activated biological glue.

To enable treating ascending aorta aneurysm without surgery as well as cerebral aneurysm, the stent graft will be implanted in folded “gear-wheel” shape and not in the regular tubular shape.

Additionally, it will be produced without shape memory metal, therefore having better flexibility to pass the aorta arch.

The stent graft will be individually tailored, implanted in a folded state, fixed at the area of aneurysms, and then expanded to its full expanded state. The stent graft radius, length and its implantation path are determined through computer simulation individually for each patient in such a way that it would fit closely patient anatomy including aneurysm area and will not penetrate aorta walls, and where such radius will have the maximal possible size subject to non-penetrating condition of given closed boundaries of aorta. The simulation is subject to non-penetrating criteria, i.e., the implantation must be done in such a way that stent will not touch the aorta walls in any point of the implantation.

A number of academic researches proved that stent graft with individual measurements provides better treatment and better chances of patient recovery.

The stent will be constructed in the folded state and it will have gear-wheel form in that state with a certain number of cogs corresponding to the aorta dimensions and aneurysm dimensions (bigger measurements require more folded state cogs).

The stent graft will be built from a biological-based material, such as collagen, having a radially compressed configuration before implantation along the longitude of the aorta and a radially expanded configuration after implantation along the longitude of the aorta.

An inside layer of stent graft having inflatable balloon like characteristics, such layer being produced and placed into the patient's body in its folded state, such layer being ballooned after stent graft implantation to the area of aneurysms to initiate stent graft expanding, such expanding performed according to the aorta measurements provided by the computerized simulation or by a physician.

The method and device of the present invention may be implemented with different types of equipment. In the following discussion, numerous specific details are set forth to provide a thorough understanding of the present invention. The stent will be made by using a number of biocompatible materials.

### BRIEF DESCRIPTION OF THE DRAWINGS

FIG. 1 provides general overview of the stent graft production process.

FIG. 2a provides an example of stent graft measurements

FIG. 2b provides graphical illustration of the stent graft shape.

FIG. 3a provides section view of the die used for tailor-made mold production.

FIG. 3b presents 3D view of the die used for tailor-made mold production.

FIG. 4a shows section view of the tailor-made mold.

FIG. 4b shows 3D view of the tailor-made mold.

FIG. 5 graphically depicted novel guidewire design.

FIG. 6 presents a section view of all stent graft layers.

FIG. 7 graphically depicts stent graft implantation procedure.

### PRIOR TO IMPLANTATION PROCEDURE

The invention will next be illustrated with reference to the figures wherein similar numbers indicate the same elements in all figures. Such figures are intended to be illustrative rather than limiting and are included herewith to facilitate the explanation of the disclosed invention.

A novel method of manufacturing stents by use of molds and dies is further presented.

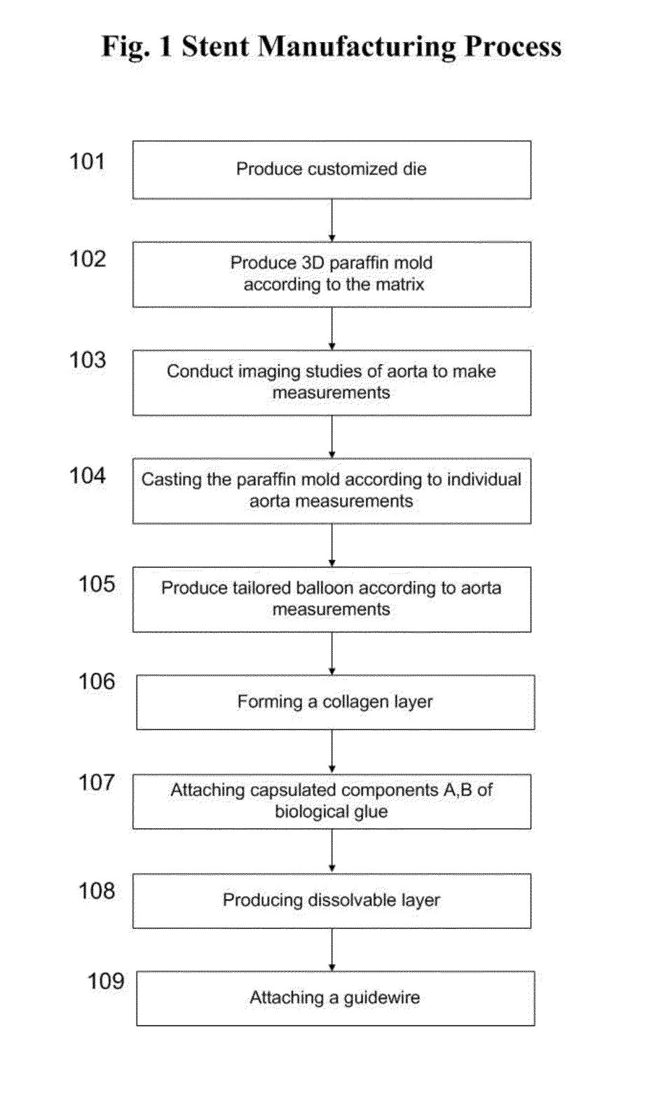

General overview of the stent graft production process is shown in the flowchart in FIG. 1.

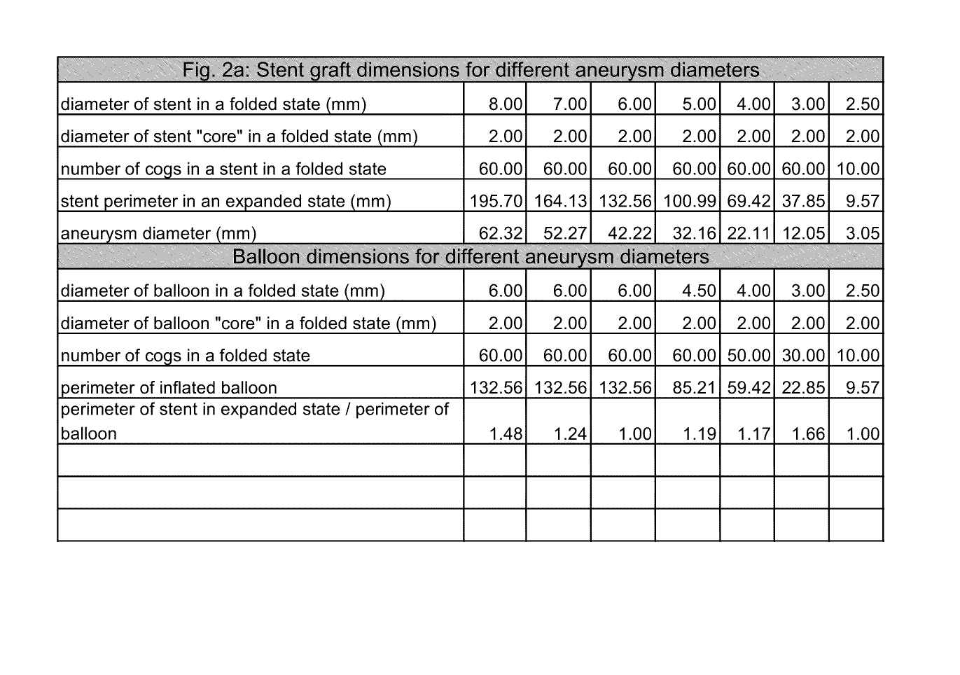

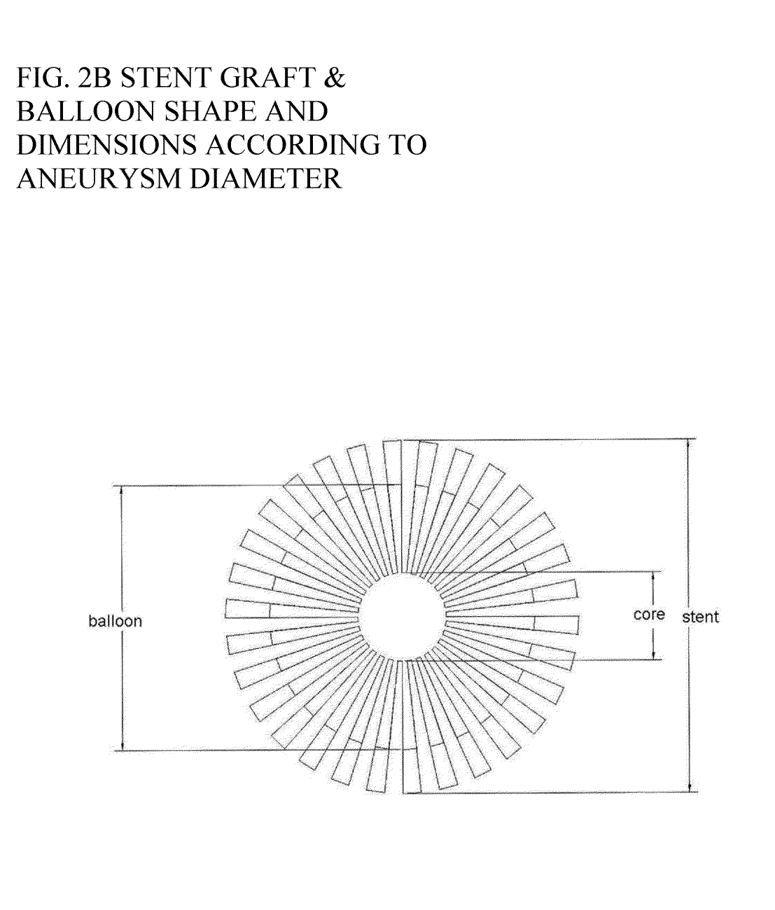

The stent graft will have gear-wheel shape in its folded state, and the balloon for the stent graft expansion will have similar type of shape. Examples of the tailor-made stent graft individual measurements shown in FIG. 2a, while the stent graft shape and the balloon shape are graphically illustrated in FIG. 2b.

a. Die

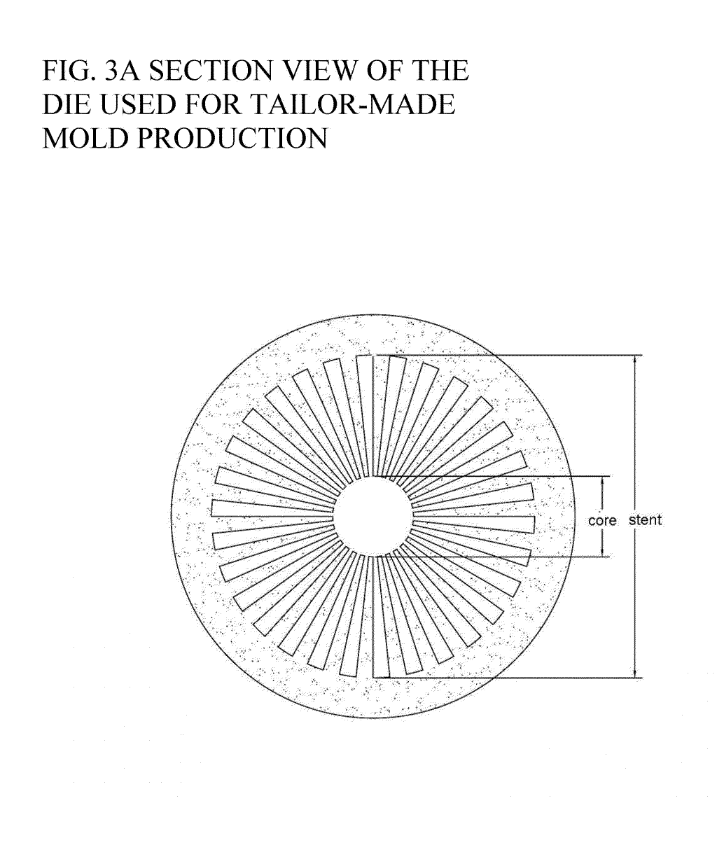

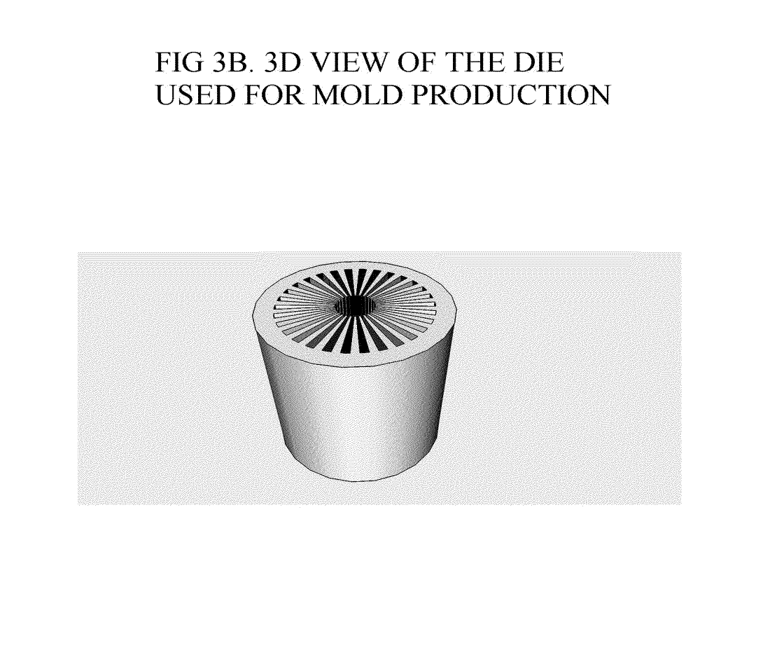

A customized manufactured die (101) will be used on the first stage of the stent graft manufacturing. The die typically is a metal block used for forming materials. The die in our invention will be primarily used for mold manufacturing. By utilizing. pressure to the die, thin section complex 3D shapes can be produced. Graphical illustration of such a die is presented in FIG. 3a, while 3D view of the die is graphically presented in FIG. 3b. Several examples of the dies are well known in the art. Manufacturing dies are typically made by tool and die makers. Dies manufacturing process produces geometrically complex parts necessary for stent graft production in our invention. Dies can be fabricated out of many different types of metals, mainly high grade tool steel and low carbon content steels. Other common materials for dies include chromium, molybdenum, nickel alloys, tungsten, and vanadium.

### b. 3D Paraffin Mold Production

The abovementioned die will be used for manufacturing of the mold further used to produce the stent graft. In the preferred embodiment of the invention, the mold will be formed from a wax-like material (102), where two types of wax could be used for the mold production. Paraffin wax refers to a mixture of alkanes that falls within the 20≦n≦40 range; they are found in the solid state at room temperature and begin to enter the liquid phase at approximately 37° C. Crude lanolin constitutes approximately 5-25% of the weight of freshly shorn wool. The wool from one Merino sheep will produce about 250-300 ml of recoverable wool grease. The wool grease is continuously removed during this washing process by centrifugal separators, which concentrate the wool grease into a wax-like substance melting at approximately 38° C.

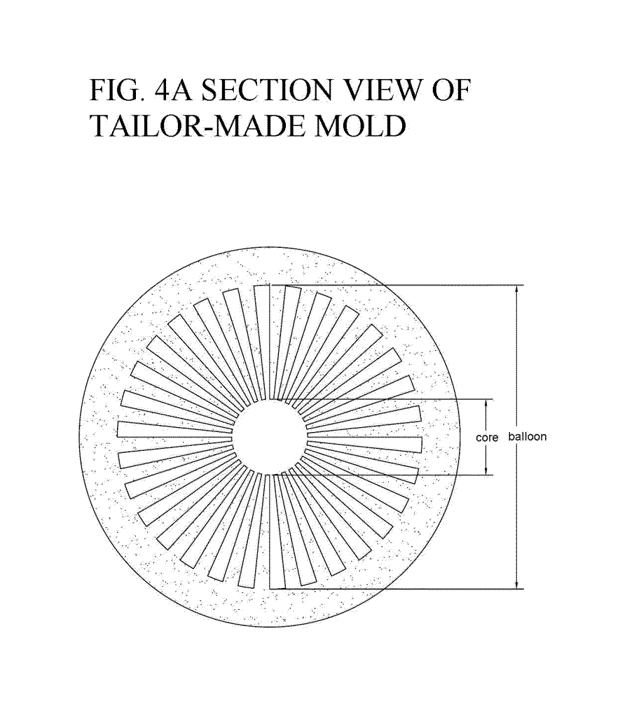

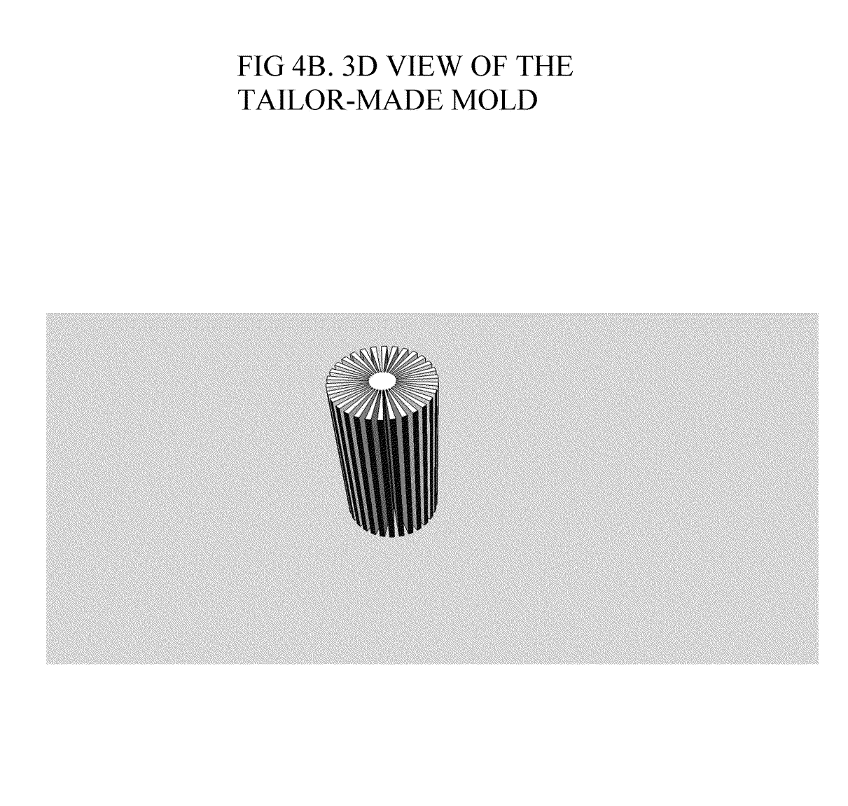

In the preferred embodiment of the invention, we use paraffin wax for mold manufacturing, the said mold being used for tailored stent graft production according to individual aorta measurement. Section view of the mold is graphically illustrated in FIG. 4a, while 3D view of the mold is presented in FIG. 4b.

c. Tailoring the Stent Graft and Pre-Procedural Imaging Studies

On the next stage, prior to the stent graft implantation procedure, a number of diagnostic imaging tests will be performed (103).

Firstly, prior to the procedure, patient's aorta measurements will be taken. According to those individual measurements, stent graft will be constructed in its folded state. So, all stent grafts will be individually tailored for each patient based on the pre-operative imaging studies. Paraffin-based mold will be “trimmed” or otherwise adjusted according to patient individual aorta measurements (104).

Because each stent-graft is custom-fitted for the patient, high quality diagnostic imaging is critical for procedural planning and determining the dimensions of the individual stent-grafts. The relationship of the aneurysm to the vessels of the head and neck and visceral circulation, the curvature of the aortic arch, and the size and tortuosity of the iliac and femoral arteries are evaluated by these different imaging.

Additionally, these diagnostic tests will also allow a physician performing the procedure to visualize the aneurysm and the surrounding area, and help him to decide on particulars of an upcoming procedure.

Before the start of stent graft implantation procedure, the physician checks and analyzes aorta images produced by MRI or other 3D medical imaging means such as X-Ray, CT, etc. in order to see and analyze aorta measurements and its structure. To do this, the physician analyzes a file obtained from 3D imaging equipment. An example of such a file could be a DICOM3 file obtained from MRI and representing aorta 3D geometry. For more comfortable image processing for some users, it can be converted from medical imaging format, such as DICOM3 to, say, one of the CAD formats, such as DXF, DWG or other. After, looking at the aorta image, the physician will see the aorta aneurysm specifics.

The stent graft measurements will be determined as defined in FIG. 2a. As detailed in the FIG. 2a, different stent graft will be produced for different types of aneurysms, when the smallest stent graft with diameter of 2.5 mm could suit the cerebral aneurysm as well. As per FIG. 2a, balloon measurements and production are the same for several measurements (clusters of measurements). In the figure, the largest stent graft will have diameter of 8 mm in the folded state and the smallest diameter in the folded state is 2.5 mm. to accommodate the cerebral aneurysm repair case.

As per FIG. 2a: lets define stent graft diameter in a folder state by a.

Lets further define stent graft “core” diameter by b.

Lets further define a number of cogs in a folded stent graft by c.

Lets now define a width of one cog (outer width) in a folded stent graft by d.

Lets further define a width of one cog (inner width) in a folded stent graft by e.

Lets further define one cog length in a folded stent graft by f.

Now lets assume that P is a perimeter of the stent graft in its expanded state and D to be an aneurysm diameter.

Then the perimeter is:

P=aπ/2+bπ/2+c(a/2−b/2)

Lets take an example from the FIG. 2a when stent graft diameter in a folded state is 8 mm, stent graft “core” diameter in a folded state is 2 mm and number of cogs is 60. Then, P=80π/2+2π/2+60(8/2−2/2)=195.7 mm.

Such a stent could be inserted inside aorta aneurysm of D=P/π=195.7/π=62.32 mm.

In terms of stent graft implantation, we will concentrate on the left boundary of the aorta image (aorta curve) and on this boundary we will connect closest points to create a line (polyline) consisting of all points of left “inside” curve of an aorta. Preferably, we would implant the stent graft alongside the closest curve of the aorta.

Total length of the stent will be pre-determined through computer simulation (prior to stent graft construction and implantation) and it will have to suit the length of aorta dimensions.

The stent graft device of this invention can also contain openings or fenestrations to allow blood flow to different arteries. This feature is especially important in cases where the aneurysm is in arch portion of the aorta. With holes or cut out areas in the device to accommodate arteries, the top of the covered section could be placed even more proximal in the aorta.

These openings or fenestrations are positioned directly in front of the origin of the arteries so as to permit blood flow into the arteries. More specifically, three openings (fenestrations) will be cut in stent graft to allow blood flow from the aorta to brachiocephalic artery, left common carotid artery and left subclavian artery. The openings will be made customary in each stent graft according to the individual measurements of aorta for each patient. That must be done in order to achieve a complete fit between three arteries measurements of the patient and three holes in the stent graft.

The proximal landing zone for the stent graft could be measured as 60 mm in diameter when extending to the area of three arteries. Three-dimensional reconstructions allow for precise measurements for creating the three fenestrations for the left subclavian artery in the stent graft. The distance from the orifice of the left carotid to the left subclavian artery is 10 mm and the distance from the orifice of the left carotid to the brachiocephalic artery is about 20 mm.

Sizes of fenestration will be adjusted individually for each patient, particularly for the patient with non-standard measurements of aorta. These openings will be made according to computer simulation of individual aorta measurements of a particular patient aorta. By default, a 10-mm fenestration will be made for left carotid and left subclavian arteries, and a 20-mm fenestration will be made for the brachiocephalic artery.

Additionally, the stent graft could be made in measurements small enough to treat cerebral aneurysm in one embodiment of the invention.

d. Stent Graft Balloon

According to the patient aorta measurements, stent graft will be folded to the dimensions needed in order for it to be placed in ascending, arch, descending or cerebral aneurysm locations.

To facilitate the stent graft expansion, the inside layer of the stent graft will have balloon like characteristics. It will be produced in the folded state, placed into the human body in that folded state, and then will be ballooned after stent graft implantation into the treatment site inside aorta walls to initiate stent graft expanding (105). The balloon will have a special gear-wheel shape in the folded state. Such a structure should enable less pressure on aorta and less chance of balloon rupturing through ballooning. By ballooning the balloon, all other layers of the stent graft will be increased therefore to fit inside aorta walls according to aorta measurements.

Inflation of the inflatable frame structure will be made through the use of a pressurized material of solid particles, gas, fluid or gel which can be injected through an injection port.

The balloon will be connected with the guidewire, the special guidewire will be a “rail” upon which the stent graft will be implanted to the aneurysm location.

Presently, most balloons are formed from a tube which is heated to above its glass transition temperature and radially expanded in a blow mold. Often, the tube is also subjected to an axial stretch so that the resulting balloon is bi-axially oriented.

In our invention, the tailor-made balloon is formed by manufacturing a balloon mold in the “gear-wheel” shape (similar to the “gear-wheel” shape of the stent graft in its folded mode) in its folded mode. Then providing a tube from medical use material (polyvinyl as an example), positioning the said tube in a precondition “gear-wheel” shape balloon mold, setting the mold measurements in accordance to the individual aorta measurements of the patient, positioning the balloon in a balloon mold, and expanding the balloon within the balloon mold to form the balloon. Radial expansion of the tube can be accomplished by heating the tube. Then the balloon will be deflated in order to its folded state.

The balloon mold is typically sized so that the balloon can be radially expanded in the balloon mold to form a balloon.

As per FIG. 2a: lets define balloon diameter in a folder state by j.

Lets further define balloon “core” diameter by k.

Lets further define a number of cogs in a folded stent graft (equal in balloon) was previously defined by c.

Lets now define a width of one cog (outer width) in a folded balloon by m.

Lets further define a width of one cog (inner width) in a folded balloon by n.

Lets further define one cog length in a folded balloon by f.

Now lets assume that PB—is a perimeter of the balloon in its expanded state.

Then the perimeter of the balloon is:

PB=jπ/2+kπ/2+c(j/2−k/2)

Lets take an example from the FIG. 2a when balloon diameter in a folded state is 6 mm, balloon “core” diameter in a folded state is 2 mm and number of cogs is 60. Then, P=6π/2+2π/2+60(6/2−2/2)=132.56 mm.

In such an example, P/PB=195.7/132.56=1.48.

The produced balloon will have characteristics such as good tensile strength and other characteristics, which are superior to the regularly manufactured balloon.

Before the procedure, balloon will then be connected to the delivery system guidewire. The balloon will be preferably positioned proximate a distal tip of the said guidewire. The stent graft, therefore, would further include delivery system for delivering and deploying a stent graft, comprising a guidewire catheter having a distal end and an interior extending along a longitudinal axis. Such a system includes a delivery guidewire, the said guidewire includes a relatively thin, flexible length of tubing. Stent delivery guidewires, particularly guidewires for delivering self-expanding stents, are also needed to exhibit tensile and/or compressive strength.

After the stent graft will be placed in the desired treatment location, the guidewire will be carefully pulled back and carefully removed from the body as a single unit.

Balloon used with our stent graft is individually tailored per patient aorta measurements and it has fewer chances to rupture due to its design as it has a special gear-wheel shape and less pressure required to inflate the balloon due to lower inflated/non-inflated balloon ratio.

d. Collagen Layer

The stent therefore can be made up of naturally occurring materials, such as collagen. Accordingly, in the preferred embodiment of the invention, material for use in the present device is collagen. The collagen layer will be accompanying the stent as a sleeve or as a tube to create an external cover layer of the stent. This layer will be placed alongside inside the aorta walls, so therefore there is a need in its biocompatible qualities of this layer material. Two layers of such collagenous material can be used to strengthen the stent structure.

Collagenous biomaterials are known due to their high blood compatibility characteristics. There are several known collagen-based materials used in medicine currently. Most medical collagen is derived from young beef cattle (bovine) or porcine (pig) tissue.

The following steps are made to form a collagen layer in a preferred embodiment of the invention (106):

placing paraffin pattern on a flat surface;

preparing liquid collagen solution;

preparing collagen and keeping collagen on ice as the collagen solidifies above 8° C.;

dipping paraffin pattern in the liquid collagen;

heating the collagen to the room temperature;

melting the paraffin by placing paraffin pattern on an electric warming table at a temperature a few degrees above the melting point of the paraffin (37° C.);

residual particles of paraffin adherent to the collagen are removed by immersing the collagen over night in xylene, a paraffin solvent.

A skilled artisan can readily adapt collagen layer forming methods to produce a collagen layer having a paraffin mold.

e. Capsulated Biological Glue

In the preferred embodiment of the invention, biological glue is used for gluing the stent graft to the aorta surface at the stent designated position, wherein the glue is remotely activated. In an aortic stent graft repair minimally invasive procedure there is an issue with gluing the stent graft to the aorta walls, as it is difficult to position the glue at the exact position where it must be administered. The problem increases in a case of ascending aorta stent graft repair, as it is increasingly difficult to pass the aortic arch and to glue the stent to the ascending portion of the aorta.

In the preferred embodiment of the disclosed invention, the biological glue is prepared prior to the start of the procedure from two main elements: each element is capsulated and covered by the capsule separately. Then, both elements will be delivered separately to the designated treatment site and mixed together at the said site, thus activating the glue. Accordingly, the components of the system will comprise capsules dimensioned and shaped to move within the body, each such capsule contains biological glue (107).

Biological glue is a natural adhesive that can be produced by a variety of ways and it is the leading surgical adhesive used in cardiovascular surgery around the world. Biological glue has been used in more than 550,000 surgical procedures since its launch in 1998. Currently there are several commercially available from-the-shelf biological glue products in the market: Evicel and Quixil are liquid fibrin sealants that contain a unique biologically active component. Other commonly commercially available bioadhesives may be used in the present invention include, but are not limited to: NEXABOND, NEXABOND S/C, and TRAUMASEAL produced by Closure Medical (TriPoint Medical); FIBRX produced by CryoLife; FOCALSEAL produced by Focal; BERIPLAST produced by Adventis-Bering; VIVOSTAT produced by ConvaTec (Bristol-Meyers-Squibb); HYSTOACRYL. BLUE produced by Davis & Geck.

In the preferred embodiment of our invention, we use bicomponent glue generated through the interaction between fibrinogen (pre-glue) and thrombin to produce fibrin (two proteins involved in the production of fibrin). Both glue components will be received in operation room facilities separately from the producer. Then, they will be capsulated separately before the surgery, preferably at the producing (plant/factory) industrial facility.

When the procedure starts, it will be placed separately at the designated treatment area location inside human body at close proximity locations. The stent graft will be sprayed with biological glue before use. After the two glue capsulated components will be placed at the designated treatment location, the glue will be activated, the adhesive components will be mixed together and the cross-linking begins. Molecules bond with other molecules, thus activating the adhesive.

The light may be applied externally activate the biological glue. Preferably, ultraviolet light or an UV laser is used to join the surfaces. The treatment site is irradiated with UV light to thereby activate the adhesive. In another aspect of the invention, the degradation of nanocapsules is enhanced by ultrasound. In still another aspect, the distribution of the nanocapsule and/or microcapsule comprising a therapeutic agent is monitored using ultrasound. Therefore, the system is presented, wherein the biological glue system further comprises a light source for activating the biological glue.

The biological glue will be capsulated in nanocapsules in the preferred embodiment of the invention. Accordingly, nanocapsules are disclosed which comprise (a) a glue-containing core and (b) a polyelectrolyte multilayer encapsulating the drug-containing core. The nanocapsules include particles whose largest dimension typically ranges between 50 nm to 10000 nm. Such nanocapsules can be prepared, for example, using various known layer-by-layer techniques, such as entail coating particles, which are dispersed in aqueous media, via nanoscale, electrostatic, self-assembly using charged polymeric (polyelectrolyte) materials. Using techniques such as those discussed above, a single glue component can be encapsulated within a single nanocapsule.

The nanocapsules and/or microcapsules of the present invention comprise a biocompatible, biodegradable polymer including polyhydroxy acid polymers such as poly-lactic-co-glycolic acid and poly-L-Lactic acid.

In one embodiment of the invention biological glue will be capsuled by nanodiamonds. Nanodiamonds can be included in various compositions of materials to take advantage of the ability of nanodiamonds to bond with biological materials and to improve mechanical strength. Nanodiamonds can be dispersed in a biologically acceptable carrier to form various nanodiamond compositions.

The compositions of the present invention can include a plurality of nanodiamond particles as a nanocapsule material. Suitable nanodiamond particles can have an average size of from about 0.5 nanometers (nm) to about 20 nm, preferably from about 4 nm to about 8 nm, and most preferably about 5 nm. Nanodiamond particles can be formed using a number of known techniques such as shock wave synthesis, CVD, etc. Currently, preferred nanodiamond particles are produced by an explosion. Aggregated clusters of nanodiamonds, ranging from 50 to 100 nm in diameter. A substantial amount of biological glue can be loaded onto clusters of nanodiamonds, which have a high surface area. Nanodiamonds possess several characteristics that make them suitable for the glue delivery as they are capable of connecting with any molecule and do not cause cell inflammation in cells once the glue will be released. Currently used materials for glue delivery can cause a serious inflammation.

Prior to the procedure, the nanodiamonds will be separately mixed with each of the two components of the biological glue, i.e. there will be a number of nanodiamonds clusters mixed with the fibrinogen, and there will be a number of clusters of nanodiamonds mixed with the thrombin. Each cluster will be separately capsulated by attaching nanodiamonds to the biological glue as a thin layer of nanodiamonds will eventually coat (capsulate) the glue. With nanodiamonds, time control of the activity of the adhesive is possible, so that the adhesive action occurs only after a predetermined time. Thus, the adhesive layer may be masked with a biodegradable protective layer, thus making it possible to prevent the adhesive power being prematurely active. The glue, loaded onto the surface of the individual nanodiamonds, is not active when the nanodiamonds are aggregated; it only becomes active when the cluster reaches its target.

Nanodiamonds coating layer may be formatted through layer-by-layer coating of the glue to produce multilayer thin films of nanodiamonds. To ensure the exact composition and thickness, the nanometer scale control could be used.

The nanocapsules can be released by applying the UV light in a preferred embodiment of the invention. The nanocapsules is actually being provided within a biodegradable coating layer that is disposed over at least a portion of the surface of the medical device, whereupon the nanocapsules are released upon degradation of the biodegradable coating layer. After allowing a sufficient time for attachment, unattached particles can be removed from the compartment prior to device removal (e.g., by vacuum), if desired, thereby limiting the systemic effects of the biological glue. Subsequent to nanocapsule attachment, encapsulated biological glue released in a controlled fashion at the site of the stent graft attachment.

The polymer-based nanocapsules or microcapsules of the present invention can be prepared in accordance with the following method. A biocompatible, biodegradable polymer is dissolved in a solution comprising an oil phase and a substance soluble in the oil phase and easy to sublime in the lyophilizer. If the oil phase is an organic solvent such as acetone, this sublimable substance may be camphor, ammonium carbamate, theobromide, camphene or napthalene. An emulsion of large beads or capsules of mixed polymer and a sublimable substance such as camphor is then formed in the solution by probe sonication. The resulting emulsion is poured into a surfactant solution, preferably a 1% solution of polyvinyl alcohol, and homogenized to remove the oil phase, for example acetone from the capsules, causing them to shrink in size. The addition of the surfactant allows the breakup of the polymer/sublimable substance beads or capsules into smaller ones, thus enhancing the size reduction of the capsules. The emulsion is then washed with deionized water to remove additional acetone and dry the capsules. The capsules are then collected by centrifugation, washed, and re-collected by centrifugation. The washed capsules are then frozen at −85.degree. C. for approximately 30 minutes and dried, preferably by lyophilization to remove any additional sublimable substance.

f. Dissolvable Layer

Next, in the preferred embodiment of the invention, the paraffin mold will be dipped again to create a dissolvable layer to be disposed as a “tunica” layer over the stent graft (108). The dissolvable layer may be placed to protect the glue component from being activated. The time it takes material to dissolve depend on the material used. A standard dissolvable polymers used in the body are Polyglycolic Acid (PGA) and Polydioxanone (PDS). These are slow dissolving materials. Sugars such as glucose can be used as a rapidly dissolving protective material.

g. Guidewire

The stent graft and the balloon will be connected to the guidewire prior to the implantation, such guidewire having a distal end tip attached to it (109). In the preferred embodiment of the invention, the guidewire will be used as a “rail” upon which the stent will be implanted to the designated treatment area. Additionally, wire-catheter having position sensors on it for an exact mapping of the aorta sizes and measurements for the exact implantation of the stent graft may be further implanted.

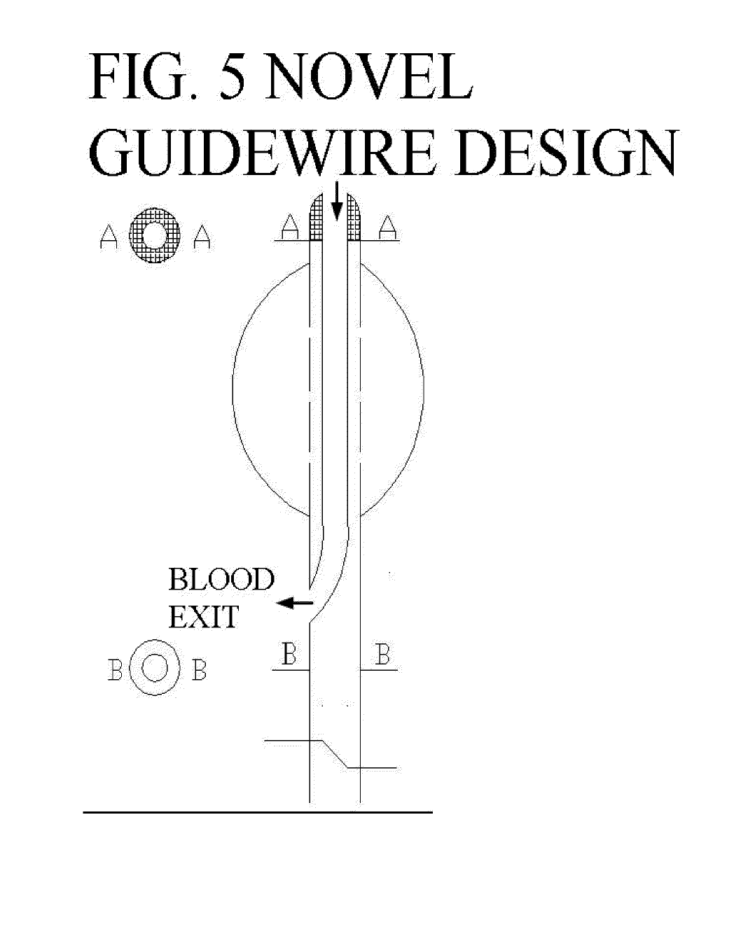

In the preferred embodiment of the invention, the guidewire will have a novel design as graphically depicted in FIG. 5. Section AA depicts a guidewire tip used for location tracking during the implantation. Such a guidewire design allows for blood flow during balloon inflating, as the guidewire will have hollow middle part of the tube (section BB) where the blood will flow. The guidewire will also have a side openings to be used for the same target.

Guidewire for the stent graft will be chosen from a number of currently available products, for example, Lake Medical Company in US produces a variety of guidewires.

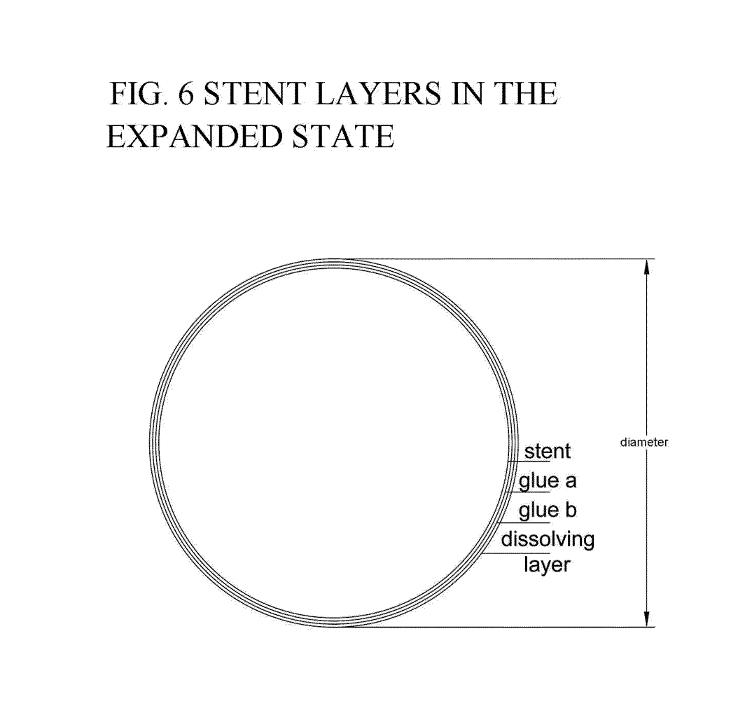

Section view of all stent graft layers is presented in FIG. 6.

### Implantation Procedure

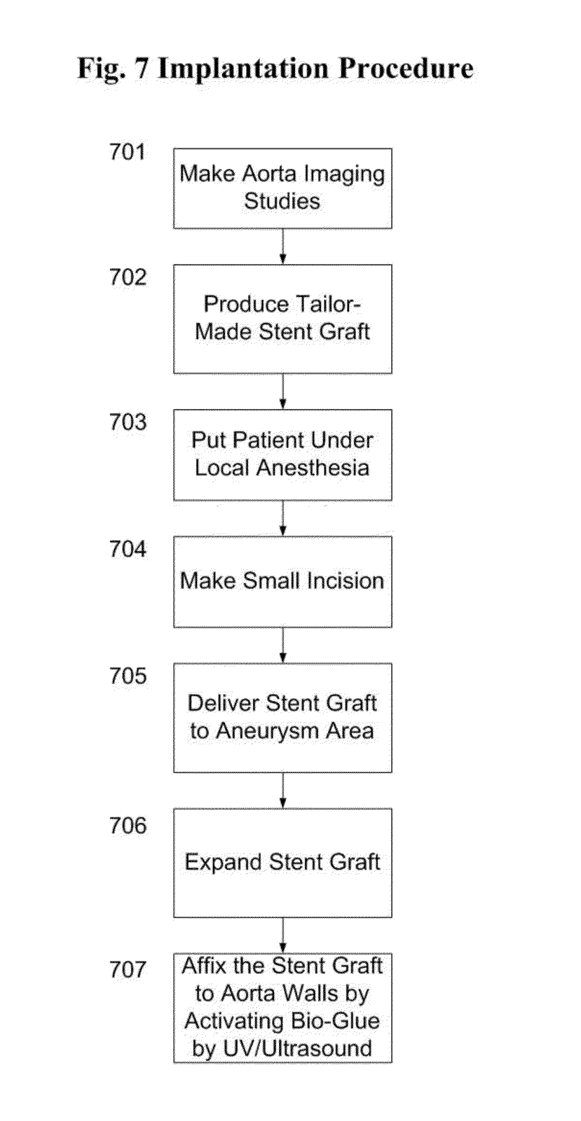

Stent graft implantation procedure is graphically depicted in FIG. 7.

As abovementioned, prior to the procedure, an image of the aorta is produced by the means of magnetic resonance imaging (MRI) or computerized tomography (CT) and the designated location of the stent graft will be determined based on the image of the aorta (701). The MRI/CT will be produced in a hospital where the patient will be further treated. Preferably, 3D imaging of the aorta will be performed with precise measurement.

Then, aorta images will be sent to the factory where the stent will be manufactured in accordance to those images and according to procedure as described above. Then, the stent will be quickly delivered to the hospital treatment site. A second stent graft will be produced to have a backup copy of the stent graft in the hospital for the same patient in case of emergency.

After stent graft will be constructed at a factory and quickly delivered to the hospital treatment site (702), the actual implantation procedure begins. The area of patient's groin where the stent are introduced will be cleaned and shaved. After that, the patient will be put under a local anesthesia 703 (or general anesthesia in cases of patients with certain medical conditions). Recent medical developments show that local or regional anesthesia is possible and even preferable in most cases, when there is no special contradictory medical evidence. The open surgery procedure requires a general anesthesia with a breathing tube and extensive intensive care unit monitoring in the immediate post-operative period, however stent graft deployment procedure can be performed under local anesthesia as well, to numb the area of the surgery and to minimize risks associated with the general anesthesia.

After the anesthesia has taken effect, the surgeon will make a small incision (704), spacing to accommodate the implant and insertion.

According to a number of researches stent graft must closely fit aorta walls, especially aneurysm area. Accordingly, there are advantages to tailor-made stent graft. Our stent graft is individually produced according to patient individual aorta measurements.

The stent graft is delivered to the area of treatment manually according to computer simulation measurements (705).

The stent graft implantation could be performed using several implantation means. In one embodiment of the invention, such implantation is MRI-guided. Using 3D real time MRI imaging, the surgeon guides the stent graft through the body to the aorta. To generate 3D images, the Magnetic Resonance Imaging (MRI) technique could be used as MRI has the ability to generate high-contrast and high-resolution images, to obtain multiple diagnostic evaluations of organ function and morphology, and to provide multiple image planes with no risk of ionizing radiation.

One and more sophisticated uses of MRI to monitor surgery in real time, called real time MRI. The real-time MRI system consists of an interactive user interface, an in-room display, specialized pulse sequences, and specialized image reconstruction software. A custom computer is connected to the commercial scanner through a gigabit Ethernet port. With this system, multiple oblique planes can be imaged and displayed simultaneously at their respective 3D locations. The rendering may be rotated on the in-room display to match the orientation of the patient, and this feature is essential for monitoring the trajectory of a device through the body. Slices may be repositioned and turned on or off as needed. MRI tissue contrast can be interactively changed by toggling saturation pulses on or off to highlight selected objects.

After making an incision and gaining access to the patient's vascular system, a guidewire tool is usually introduced and guided under visualization to the intended place in patients' body. The guidewire then serves as a “rail” upon which other subsequent devices are guided through the vessels. Stent graft will then be deployed over that special guidewire. Every step of the introducing system advancement will be controlled by using real time MRI images. The cross-section of the stent has gear wheel shape to allow the blood flow during the stent implantation and balloons inflation.

Next, the stent graft will be advanced through that incision to the aorta. In one embodiment of the invention, the stent graft implantation path will be determined using computer simulation using trial-and-error method. As a result of this computer simulation we obtain a sequence of points for implanting the stent graft in a folded state {(Xₖ^{S}, Yₖ^{S}, Zₖ^{S})}ₖ₌₁^{N}.

In another embodiment of the invention, stent graft implantation will be done robotically and/or with using magnetic tracking of the stent graft, and stent graft tip in particular. The robotic features are used in our invention in order to have an option of implanting the stent graft into its designated place at aorta with the use of robot and, thus, to avoid manual surgeon work which can be less accurate than the robotic work. Accordingly, the invention further includes robotic means under the control of the user for generating and implementing a preprogrammed optimal path of moving the stent graft into its treatment site, and for automatically carrying out the robotic-executed surgery without active participation by the user during the procedure. This can be done by moving the stent through aorta by manipulating electromagnets with robotic means or by manually manipulating magnets with some sort of controller device such as a joystick, a mouse or a keyboard. In case of a robotic implantation, a special detector will be added to determine the orientation of the master unit and the orientation of the slave unit. To implement that, it is necessary to precisely determine the position (spatial position and sometimes the angular position) of the distal tip of the stent graft. General purpose instruments have been developed that incorporated bend and twist sensors distributed along their length at known intervals. These bend and twist sensors allow the user to approximate the tip position of the device by monitoring the manner in which the device moves as it is progresses in 3D space. A sensor data processing system is coupled to these bend and twist sensors and receives the flexure signals from these sensors. The processing system monitors the bend and twist sensors disposed along the device and extrapolates the device and tip position. This type of a system is known as a path-dependent measuring system; i.e., a system that requires knowledge of the spacing between each pair of sensors and a signal from each sensor to perform an extrapolation to determine the device's orientation. Particularly important is that, for path-dependent systems such as this, the distal end tip position is determined successively from intermediate measurements along the length of the flexible structure, beginning at a known location, typically the proximal end. Currently there are a number of six degrees of freedom sensors available on the market that can be suitable for our needs. For example, there are six degrees of freedom sensors in Aurora Measurement System by NDI company. The Aurora tracks miniaturized sensors designed for integration into surgical tools and instruments, such as catheters or guidewire, wherein Aurora sensors are placed at the tip of such catheters or guidewire to allow for the localization of the object located inside the body.

An additional advantage in using robots is in the fact that they can use their many internal degrees of freedom to thread through tightly packed places accessing locations that people and machinery otherwise cannot use. Moreover, some sophisticated robots can coordinate their internal degrees of freedom to perform a variety of locomotion capabilities.

In preferred embodiment of the invention, the stent graft implantation assembly will comprise:

stent graft implantation means as described in a more details in paragraphs above;

expansion mechanism to remove the support assembly from the stent graft, permitting subsequent radial expansion of the stent after it has been placed in the desired location;

a detector for determining the location or orientation of the stent within the body. The detector can be selected, for example, from devices utilizing a number of different techniques such as x-ray analysis, ultrasonic sensing, magnetic position sensing. The stent location tracking could be achieved in a number of ways known in the art, including electromagnetic tracking and/or sensor-based tracking. For example, Medtronic company developed radiopaque markers-based system to facilitate precise stent graft delivery. Such markers are sewn to the graft to help visualize and identify the following the location of the stent graft. Electromagnetic tracking of the stent graft deployment could be done by advancing a tracked guidewire made of coil to the aorta and positioning the tracked stent-graft assembly by using electromagnetic guidance. Multiple MRI scans could be obtained to evaluate the accuracy of the electromagnetic tracking system by displaying “virtual” electromagnetic-tracked position. An another example of electromagnetic tracking system is Aurora Electromagnetic Measurement System by NDI company.

The Aurora tracks miniaturized sensors designed for integration into surgical tools and instruments, such as catheters or guidewire, wherein Aurora sensors are placed at the tip of such catheters or guidewire to allow for the localization of the object located inside the body.

there will be a number of magnets manipulated by robotic means in such a way that at least one of the magnets will be placed above the patient's body and at least one of the magnets will be placed below the patient's body.

To ensure optimal stent graft placement into the aorta, we perform a computerized simulation of placement for each interval of inner polyline of the stent. Obviously, stent graft should not penetrate aorta walls, therefore in such computer simulation, the stent measurements will have there maximal possible size subject to non-penetrating condition outside of given closed boundaries of aorta.

To implant the stent graft using robotic means, we have to calculate the path that will be used as an input for a robot. For that, we will create a mathematic model that calculates sequence of points for robotic movement. The movement of an object by electromagnetic forces will be done by changing the electrical current that passes through the wires wrapped around an electromagnet. Electromagnets use electric current to generate a magnetic field which can be turned on or off as needed.

Stent graft implantation is carried out by a number of pulses (moves). Lets take a sequence of points {(Xₖ^{S}, Yₖ^{S}, Xₖ^{S}), k=1, 2, . . . , K} for implantation of aorta in the folded state received by the trial-and-error computer simulation method where K represents a total number of pulses of the stent graft from its insertion to the completion of pulses to the destination point at the treatment location, and where k represents a number of current move.

Lets define this set of stent graft positions by {(Xₖ^{C}, Yₖ^{c}, Zₖ^{C}), k=01, 2, . . . , K}, where K is a total number of pulses and k is a number of current pulse.

Additionally, let's define stent graft weight (mass) as M.

Now, we assume that there will be a number of magnets in the model (at least one of them directed above the patient's body and, at least, one another one below it). Let's now assume that every magnet will have its own specific strength. Let's define strength of a specific magnet by (F_{S}). The magnetic strength of an electromagnet depends on the number of turns of wire around the electromagnet's core, the current through the wire and the size of the iron core. Increasing these factors can result in an electromagnet that is much larger and stronger than a natural magnet.

What we want to obtain as an output of this mathematic model is a sequence of 3D points of magnets positions where the implantation could be carried out by the use of 1 or 2 magnets.

In case of implantation by the use of two magnets, this sequence is defined by

S₂={(Xₖ^{Q},Yₖ^{Q},Zₖ^{Q})}ₖ₌₁^{N}, Q=1,2;

where Q is a number of electromagnet (1^{st} and 2^{nd}) and k is a number of current point of the sequence. N represents a number of consecutive positions of magnets required for implanting the stent graft into the treatment site.

In case of robotic implantation by the use of one magnet, this sequence is defined by

S₁={(Xₖ,Yₖ,Zₖ)}ₖ₌₁^{N};

where k is a number of current point of the sequence and N represents a number of positions of magnets required for implanting the stent graft into the treatment site. Additionally, as an output of this model, for each point of the sequence above we calculate strength of electromagnets at that particular point.

Let's define such strength of electromagnets' strengths as {Fₖ^{Q}}ₖ₌₁^{N}, Q=1,2, where N represents a number of positions of magnets required for implanting the stent graft (or a number of pulses).

In addition to abovementioned factors, we have to take in consideration a specific electromagnetic capacity (inductance) of such magnetic tip as the abovementioned robotic means will move the stent graft with a magnetic tip placed in the head of it.

Additional factor to be accounted is a different permeability of the air and of the human body. Let's define them as μ_{A}, μ_{B} accordingly.

As stent graft will be implanted along the aorta walls a special friction factor has to be determined in regard to a friction of the stent graft with aorta walls. For that, let's define friction coefficient f.

Now, after defining all factors pertaining to the model, let's compute a sequence of points. For computing this sequence of points, let's define a local coordinate 3D system with a unit equal to 1 millimeter as follows:
The start of this system will be in the low-left corner of the table the patient is laid on (i.e., an MRI table)
The X axis will be directed in left to right direction, as usual, and the Y axis will be directed in down-upwards direction, as usual in system of coordinates, and the Z axis will be directed from the table in upwards direction

The stent graft implantation will be carried out by discrete pulses. In order for magnet to move the object by discrete pulses, the strength of magnetic horizontal projection forces which we define by S_{H}^{M} has to be equal or higher that the friction strength S^{F} inside aorta, when the friction strength depends on the stent mass and friction coefficient (f).

To formulate this condition for implantation by use of two magnets, let's define

S₂^{Result} as a net strength required for moving the stent graft

M was already defined as a total weight (mass) of the stent graft;

a is an acceleration that is sufficient to move the stent for required distance D (e.g. 1 mm) during reasonable time interval t (e.g. 1 sec), since:

at²/2=D or a=2D/t²

Accordingly, we define this condition by:

S₂^{Result}=S_{H}^{U}+S_{H}^{D}−S^{F}=Ma

Lets t=1 sec, D=0.0005 m

then the moving condition is in a case of single magnet will be [a1]

S^{M} cos α_{T}^{M}/δ_{T}²−[(Km+m₀)g−S^{M} sin α_{T}^{M}/δ_{T}²]f=0.001K,m

where

K is a number of pulses in a stent;

m is a weight (mass) of each sector;

m_{T} is mass of the magnetic tip mounted on the top of the stent;

g=9.8 m/sec² is an acceleration of free fall;

S^{M} is magnetic strength between the magnet and the tip at the distance 1 meter;

α_{T}^{M} is the angle between direction from the tip to the magnet and system of coordinates plane;

δ_{T} is the distance from magnet to the tip;

The moving condition in a case of two magnets (one above and one below the surgery table) and if magnetic influence of rings is negligible [a1] will be:

S^{T} cos α_{T}^{U}/δ_{U}²+S^{T} cos α_{T}^{D}/δ_{D}²−[(Km+m₀)g−S^{T} sin α_{T}^{U}/δ_{U}²+S^{T} sin α_{T}^{D}/δ_{U}²]f=0.001Km

where

α_{T}^{U} is an angle between direction from the tip to the upper magnet and system of coordinates plane;

α_{T}^{T} is an angle between direction from the tip to the down magnet and system of coordinates plane;

δ_{U} is the distance from the upper magnet to the tip;

δ_{D} is the distance from the down magnet to the tip;

In a case of single magnet decision variables are:

α_{T}^{M}: the angle between direction from the tip to the magnet and system of coordinates plane;

δ_{T}: the distance from magnet to the tip.

The missing angle β_{OX} from OX axis from inside the plane is received from the required path {(Xₖ^{S}, Yₖ^{S}, Zₖ^{S}), k=1, 2, . . . , K} of the stent graft tip as computed by of computerized simulation.

$${β}_{OX} = arctan \frac{{Y}_{k}^{S} - {Y}_{k - 1}^{S}}{{X}_{k}^{S} - {X}_{k - 1}^{S}}$$

in a case that denominator Xₖ^{S}−Xₖ₋₁^{S}≠0, else β_{OX}=π/2.

Separation of decision variables gives the following:

$$\frac{{S}^{M}}{{δ}_{T}^{2}} ( cos {α}_{T}^{M} + f sin {α}_{T}^{M} ) = 0.001 Km + ( Km + {m}_{T} ) gf$$

For the distance from magnet to the tip:

$${δ}_{T} = \sqrt{\frac{{S}^{M} ( cos {α}_{T}^{M} + f sin {α}_{T}^{M} )}{Km ( gf + 0.001 ) + {gfm}_{T}}}$$

$$Let {α}_{0} = π / 3 = 60 °$$

$$then$$

$${δ}_{T} = \sqrt{\frac{{S}^{M} ( 1 + 1.732 f )}{2 [ {m}_{T} gf + Km ( 0.001 + gf ) ]}}$$

For example:

$${S}^{M} = 0.01 Newton ;$$

$$f = 0.1 ;$$

$$m = 0.01 kg ;$$

$$g = 10 m \text{/} {s}^{2} ;$$

$$K = 10 ;$$

$$\begin{matrix}{δ}_{T} = \sqrt{\frac{0.01 \cdot 1.1732}{2 ( 0.01 \cdot 10 \cdot 0.1 \cdot + 10 \cdot 0.01 \cdot 1.001 )}} \\ = \sqrt{\frac{0.0117}{0.22}} \\ = \sqrt{0.05} \\ = 0.22 m \\ = 22 cm .\end{matrix}$$

In a case of two magnets decision variables are:

α_{T}^{U}: the angle between direction from the tip to the upper magnet and LCS plane;

α_{T}^{D}: the angle between direction from the tip to the down magnet and LCS plane;

δ_{T}^{U}: the distance from the upper magnet to the tip.

δ_{T}^{D}: the distance from the down magnet to the tip.

Separate variables of upper and down magnets:

$$\frac{{S}^{T}}{{δ}_{U}^{2}} ( cos {α}_{T}^{U} + f sin {α}_{T}^{U} ) + \frac{{S}^{T}}{{δ}_{D}^{2}} ( cos {α}_{T}^{D} - f sin {α}_{T}^{D} ) = Km ( 0.001 + gf ) + {m}_{T} g$$

$$Or$$

$$\sqrt{1 + {f}^{2}} [ \frac{{S}^{T}}{{δ}_{U}^{2}} cos ( {α}_{T}^{U} - ϕ ) + \frac{{S}^{T}}{{δ}_{D}^{2}} ( cos ( {α}_{T}^{D} + ϕ ) ] = Km ( 0.001 + gf ) + {m}_{T} g \\ where \\ ϕ = arccos \frac{1}{\sqrt{1 + {f}^{2}}} ;$$

Because friction coefficient f<0.1 then φ≈0, therefore if suppose α_{T}^{U}=α_{T}^{D}=α_{T} hence:

$$1 / {δ}_{U}^{2} + 1 / {δ}_{D}^{2} = \frac{Km ( 0.001 + gf ) + {m}_{T} g}{{S}^{T} cos {α}_{T} \sqrt{1 + {f}^{2}}}$$

For example:

$${S}^{M} = 0.01 Newton ;$$

$$f = 0.1 ;$$

$$m = 0.01 kg ;$$

$$g = 10 m \text{/} {s}^{2} ;$$

$$K = 10 ;$$

$${α}_{T} = π / 3 = 60 ° ;$$

$$Then$$

$$1 / {δ}_{U}^{2} + 1 / {δ}_{D}^{2} = \frac{10 \cdot 0.01 \cdot ( 0.001 + 10 \cdot 0.1 ) + 0.01 \cdot 10}{0.01 \cdot 0.5 \sqrt{1 + 0.01}} = 40$$

$$Let {δ}_{U} = 0.2 then 1 / {δ}_{D} = \sqrt{15 } since {δ}_{D} \approx 0.26 = 26 cm .$$

Following implantation, the stent graft is then expanded (706) to sit snugly inside the artery and it provides a reinforced channel for the blood to flow and thereby reduces the pressure on the damaged area (aneurysm) of the artery. This, in turn, prevents the aneurysm from rupturing.

To facilitate stent graft expansion, the inside layer of the stent graft will have balloon like characteristics. It will be produced by inflating and then folding it again, placing into the human body in that folded state, and then will be ballooned after stent graft implantation into the treatment site inside aorta walls to initiate stent graft expanding. Accordingly, the balloon will be ballooned to its maximal state necessary to unfold the stent graft inside aorta walls according to the measurements provided by the computerized simulation based on data from MRI or based on CT measurements or by the physician.

After stent graft will be enlarged radially, it will remain in place by affixing it to the vessel wall. Generally, speaking there are two most commonly used methods to fix the stent graft in its place. The first is by affixing special anchoring surgical barbs at the both ends of a stent graft. It can be affixed to stent graft's outside layer by soldering, brazing, or welding to the shape memory metal part of the stent graft. Attaching of the stent graft to the implantation site will then be done by inflating a balloon in order for stent to be fully expanded and to press against the aorta walls and by seating the barbs of the stent into the wall.

The second method (this method is used in a preferred embodiment of the invention) is by using various types of medical grade adhesives. These products can be activated by exposure to UV light/ultrasonic energy (707).

In the case of ultrasound excitation, the capsules are preferably doped with magnetite nanoparticles, nanodiamonds, silica nanoparticles, ceramic nanoparticles or similar particles which absorb the ultrasound energy. The capsules also can be doped with polymers susceptible to the exerted force.

After finishing the stent graft placement procedure, the wound made by incision would be closed by the use of sutures. A suture is any strand of material used to approximate tissue or ligate vessels. Currently, sutures are classified to the absorbable or non-absorbable types based on their absorptive properties.

Absorbable sutures are removed from the body by enzymatic action or hydrolysis. All absorbable sutures eventually completely dissolve. Absorbable sutures are not recommended for patients with fever, infection, or poor nutritional status, absorption of absorbable suture may accelerate and lead to premature diminution of tensile strength. Non-absorbable sutures are composed of multiple filaments of metal, synthetic fibers, and organic fibers. Non-absorbable sutures needed to be removed within a time specified by the doctor.

After the procedure, the patient will be monitored carefully by the doctors. MRI imaging and/or X-rays and/or ultrasound imaging will be proceeded to make sure that the stent graft is properly placed and occluding blood flow to the aneurysm.

Recovery time varies by the patient. The patient typically must stay in the hospital after the procedure for a couple of days. There may be some discomfort after the procedure and it might be required to lie flat for several hours to allow the wounds to begin healing. The patient may also experience side effects such as swelling of the upper thigh, numbness of the legs, nausea, vomiting, leg pain or throbbing, malaise, lack of appetite, fever for several days after the operation. Blood sampling may also be performed for several times after the procedure. In most cases, patients return to normal activities within 4 to 6 weeks. The patient will also receive instructions about what to eat and do before and after the procedure. In cases of increased pain, swelling, redness, red streaking or separation of the wound, the patient has to contact the doctor immediately.

The stent graft is meant to remain in patient's body for the rest of the life, so there is a need for regular post-procedure appointments with the physician to make sure the stent graft is working properly.

### Additional Embodiments of the Invention

### Drug Delivery

The stent graft can also provide a localized pharmacological treatment of a vessel. In particular, the stent graft in disclosed invention could also be used as a delivery tool to deliver anti-plague drugs (cholesterol lowering drugs) to the aorta. To enable such qualities, anti-plague drugs will be injected into the stent graft. Drugs like lovastatin, pravastatin, rosuvastatin, simvastatin and statin drugs are very effective for lowering cholesterol levels.

The chosen drug could be mixed or bound to a stent graft coating prior to stent graft implantation procedure. Alternatively, micro pores or nanopores might be produced in the stent to facilitate drug elution.

### THE STATE OF ART AND BACKGROUND OF THE INVENTION

Over 6 million people in the developed world are currently living with an aortic aneurysm and every year 750,000 new cases are diagnosed. This number is increasing rapidly due to improved imaging technology and better screening protocols.

The stent graft market is relatively new and is currently driven by new device approvals in key markets. Over the past five years the market has been growing at a compound annual average growth rate of 70% as recently there has been a focus on using minimally invasive approaches in cardiac surgery in an effort to reduce trauma and increase speed of recovery for the patient. Particular emphasis has been made on endovascular repairs, and especially, no-cut aorta aneurysm repairs.

The incidence of thoracic aortic aneurysms (TAAs) and acute dissections is estimated to be as high as 10 cases per 100,000 people per year giving a potential number of 30,000 cases per year in the US and 48,000 cases in Europe. An estimated 35,000 thoracic aortic surgical and endovascular interventions are performed every year in the developed world (10,000 in the US alone) at a combined cost to individuals, insurance companies and tax payers of $3.5 billion a year.

Surgical repair in this anatomical region is difficult and has been associated with devastating complications, especially in those who are not fit for thoracotomy. Rupture of TAA has a mortality rate of 97%, and a median survival rate of 3 days. Annual mortality from ruptured aneurysms in the United States is about 15,000.

In selected patients, a stent graft advantageously eliminates the need to perform open thoracic or abdominal surgical procedures to treat diseases of the aorta and eliminates the need for total aortic reconstruction. Thus, the patient has less trauma and experiences a decrease in hospitalization and recovery times. The time needed to insert a stent graft is substantially less than the typical anesthesia time required for open aortic bypass surgical repair, for example. Currently, over 35% of aneurysms treated with stent grafts. The endovascular approach can potentially replace many operations. Furthermore, the endovascular approach can double the current universe of eligible patients, since many diagnosed cases are managed conservatively due to patient non-eligibility for surgery. Open surgery is fraught with high morbidity and mortality rates, primarily because of the invasive and complex nature of the procedure. Complications associated with surgery include, for example, the possibility of aneurysm rupture, loss of function related to extended periods of restricted blood flow to the extremities, blood loss, myocardial infarction, congestive heart failure, arrhythmia, and complications associated with the use of general anesthesia and mechanical ventilation systems. In addition, the typical patient in need of aneurysm repair is older and in poor health, facts that significantly increase the likelihood of complications.

One example of endovascular graft for treating only descending and abdominal aorta aneurysm is a Zenit TX2 and Zenit Flex endovascular graft series by CookMedical company. One further example of endovascular stent graft for treating descending and abdominal aneurysm is presented in U.S. Pat. No. 7,404,823 by Gregorich from Boston Scientific company. Another examples of such endovascular grafts/prosthesis are disclosed in US patent application 20060287714 by Erbel and US 20060287713 patent application by Douglas as well as in U.S. Pat. No. 7,175,651 by Kerr, U.S. Pat. No. 5,928,280 by Hansen and U.S. Pat. No. 5,100,429 by Sinofsky.

Using minimally invasive endovascular techniques, the Merci retrieval procedure is performed in a hospital angiography suite by a trained physician. The Merci Retriever is inserted through the femoral artery and advanced into the cerebral vasculature using fluoroscopic or X-ray imaging to pinpoint clot location. Once the Merci Retriever is at the site of occlusion, the physician performs the standard Merci procedure. The Merci Retriever works with the outreach distal access catheters work with the Merci retrieval system to aid in clot removal. The distal access catheter provides increased support and changes the force vector to be in line with the clot face.

There is also a AngioJet Ultra Thrombectomy System providing similar thrombectomy treatment. This system actually is a combination of catheter based systema and it also has a vacuum feature for clot retrieval as clots are vacuumed away from the body.

http://www.springerlink.com/content/9tvgbpc9v9xr1p3j/

http://www.thefreelibrary.com/New+catheter+technique+less+invasive+and+risky+than+age-old+brain+...-a0212351342

In respect to MRI-guided aspect in out invention, An example of the real time MRI monitoring system is described at http://www.pubmedcentral.nih.gov/articlerender.fcgi?artid=1963465. This system is working by recording a reference signal from the rotating permanent magnet synchronously with the rotating magnetic dipole. This article represents a new generation of short (120 cm), wide-bore (70 cm) 1.5 T imaging systems (Magnetom Espree, Siemens Medical Solutions) has recently been introduced. This magnet design gives a clearance of up to 30 cm above the chest of the supine patient, and the short design allows a surgeon to directly manipulate thoracoscopic instruments within the chest with ample “attack angles” and degrees of freedom. The more open bore allows better access to the patient for anesthesia when imaging the heart. The imaging gradients and amplifiers of the new systems yield a scanning performance that rivals that of the standard cardiac MR scanners, and therefore high-quality images can be obtained with real-time acquisition speeds. The excellent blood/tissue contrast and the ability to interactively adjust imaging planes to view devices and the beating heart from multiple simultaneous viewpoints makes real-time MRI ideal for guiding cardiac surgical interventions.

Still, only stent graft for treating abdominal and descending aorta aneurysms has been currently developed and in this invention we disclose a stent graft for repairing arch and ascending aorta aneurysm also. An example of endovascular graft for treating descending and abdominal aorta aneurysm is a Zenit TX2 and Zenit Flex endovascular graft series by CookMedical company. One further example of endovascular stent graft for treating descending and abdominal aneurysm is presented in U.S. Pat. No. 7,404,823 by Gregorich from Boston Scientific firm. Another examples of such endovascular grafts/prosthesis are disclosed in US patent application 20060287714 by Erbel and US 20060287713 patent application by Douglas as well as in U.S. Pat. No. 7,175,651 by Kerr, U.S. Pat. No. 5,928,280 by Hansen and U.S. Pat. No. 5,100,429 by Sinofsky.

U.S. Pat. No. 8,043,354 by Greenberg titled “Thoracic deployment device and stent graft” presents a stent graft whereby control of the stent graft can be maintained while allowing access into the lumen of the stent graft

Another US patent by Greenberg is U.S. Pat. No. 8,002,816 is titled “Prosthesis for implantation in aorta and method of using same”. U.S. Pat. No. 8,002,816 presents a prosthesis for implantation in the ascending aorta comprising a tubular body made of biocompatible graft material, a cuff at the proximal portion of the tubular body for biasing pressure onto a sino-tubular junction and that is configured to conform to the junction. Still, this invention relates to the treatment of one form of aortic aneurysm known as an aortic dissection in the ascending thoracic aorta.

Magnetic and robotic methods of performing endovascular procedures have been disclosed in a number of prior art references. US 20120065467 by Moll titled: “Robotic Catheter System and Methods” discloses a robotic endoscopic instrument system that includes an operator control station located remotely from an operating table, to which a instrument driver and instrument are coupled by a instrument driver mounting brace wherein the communication link transfers signals between the operator control station and instrument driver. Magellan robotic system by Hansen Medical has been cleared by the FDA for peripheral vascular interventions.

Northern Digital company is an owner of a number of prior art patents and patent applications in field of magnetic tracking systems with microsensors. Two of those are US 20020052546 “Flexible instrument with optical sensors” and US 20080107305 “Integrated mapping system”.

Blume, in his U.S. Pat. No. 6,014,580 discloses device and method for specifying the orientation of a magnetic field produced in a patient to aid surgical procedures involving an implanted magnet.

One more similar field invention by Shahar is WO/2004/006795 named: “Apparatus For Catheter Guidance Control And Imaging” in which Shahar discloses a system similar to his U.S. Pat. No. 7,280,863.

Another variation of magnetic based system for moving catheters or other interventional devices inside the human body was presented in the article Modeling magnetic catheters in external fields by Tunai (http://www.ncbi.nlm.nih.gov/pubmed/17272111).

Yet another patent in the field of motion control using electromagnetic forces is disclosed in U.S. Pat. No. 7,348,754 by Prasanna. This invention describes an apparatus comprising two basic electromagnetic components, wherein in both such components there are one or a few electromagnetic members coupled in such a way that the second component moves with respect to the first component in a cyclical manner by interaction one or more electromagnetic members of the first component interacts with the one or more electromagnetic members of the second component during each of one or more cycles of motion of the second component with respect to the first component such that, when a constant force profile is applied to move the second component with respect to the first component.

None of these patents don't disclose a system for moving stent graft or catheters with the use of magnets controlled by robots but all of them are controlled by the surgeon performing the procedure.

With respect to the biological glue aspect of the disclosed invention, there are a number of medical applications, as well as patents and patent applications that use biological glue for gluing the stent to the aorta.

Currently there are several known medical applications for biological glue with many more potential uses to come in the future. Biological glue can be currently used for a variety of medical applications. It is used for the control of pulmonary air leaks. In selective intrabronchial tamponade the glue is instilled into the bronchial tree through a flexible bronchoscope, and in therapeutic pleurodesis it is instilled into the pleural cavity through a chest drainage tube.

In addition two new methods of using biological glue have been developed for the control of persisting air leaks. In selective intrabronchial tamponade the glue is instilled into the bronchial tree through a flexible bronchoscope, and in therapeutic pleurodesis it is instilled into the pleural cavity through a chest drainage tube. The air leaks were resolved in all cases. Therapeutic pleurodesis alone was successfully carried out in one patient and as an adjunct to selective intrabronchial tamponade on two occasions. A thoracotomy was eventually needed in one of the seven patients.

Additionally, biological glue was used in transanal advancement flap repair in the treatment of high transsphincteric fistulas with instillation of biological glue improving the healing rate following transanal advancement flap repair for high transsphincteric fistulas.

Additionally, fibrin-based sealants are frequently used to reduce blood loss during/after surgery. The sealants, formed by mixing a concentrated solution of fibrinogen with thrombin and Ca.sup.2+ to produce fibrin, are applied to bleeding wounds and suture lines to help stop bleeding.

Biological glue by Cryolife company is the leading surgical adhesive used in cardiovascular surgery around the world. Biological glue is composed of purified bovine serum albumin and glutaraldehyde. The two glue components are dispensed by a controlled delivery system comprising a double-chambered syringe, applicator tips, and optional extender applicator tips. The glue begins to polymerize within 20 to 30 seconds and reaches its bonding strength within two minutes.

An Omnex surgical sealant has been used in cardiac surgery. Omnex is a synthetic sealant that forms a physical barrier to mechanically seal tissue and block the passage of blood, body fluids or air. Research data suggests that it is an effective surgical sealant that is able to seal anastomosis, to reinforce and to achieve hemostasis along critical suture lines in cardiothoracic surgery.

Evicel is a fibrin glue manufactured by OMRIX Biopharmaceuticals LTD in Israel, has been approved by FDA for use for general hemostasis in surgery. Evicel provides a new option to help control bleeding during general surgery, when other approaches and techniques are ineffective or impractical. Once applied, it forms a covering that helps stop bleeding. Evicel contains two main elements: fibrinogen and proteolytic enzyme called thrombin, two proteins involved in the production of fibrin. Fibrinogen and thrombin are found in human plasma, and the plasma used to manufacture the product is collected from donors who have been screened and tested for blood-transmitted infections. The fibrinogen and thrombin also undergo a two-step process to reduce the risk for the transmission of potentially contaminating bloodborne viruses. Such glue described in U.S. Pat. No. 6,019,993 by Bal assigned to OMRIX Biopharmaceuticals.

Allen in US Patent Application 20090312743 titled “Perivascular Leak Repair System” discloses the perivascular leak repair system which provides a sealant reservoir with a repair catheter operably attached, and in particular, a method of sealing a perivascular leak by identifying such perivascular leak, inserting a repair catheter to the perivascular leak, injecting sealant at the perivascular leak and removing the repair catheter.

Shriver in U.S. Pat. No. 7,771,442 discloses graft core for seal and suture anastomoses with devices and methods for percutaneous intraluminal excisional surgery—a combination anastomosis device that both sutures and seals connections between two native body tubes and a graft—better proof against leaks than prior art of suturing alone or as some propose, by sealing.

Popov in his U.S. Pat. No. 6,068,637 titled “Method and devices for performing vascular anastomosis” discloses a method and devices are provided for performing end-to-side anastomoses between the severed end of a first hollow organ and the side-wall of a second hollow organ utilizing transluminal approach with endoscopic assistance, wherein the first and second hollow organs can be secured utilizing a biocompatible glue, clips or by suturing.

U.S. Pat. No. 7,851,447 by Muir titled: “Methods for nerve repair” discloses a method for repairing damaged nerve tissues where tissue adhesive used is a biological glue, wherein the biological glue is a fibrin-containing adhesive, such as fibrin glue, fibrin sealant, or platelet gel.

Buratto in his U.S. Pat. No. 5,935,140 “Method for modifying the curvature of the cornea” discloses a surgical method for modifying the curvature of the cornea for the correction of ametropias wherein the superficial layer is glued onto the underlying surface with a biological glue.

Alio Sanz in his 20090317483 “Bicomponent Bioadhesive for Biomedical Use” teaches new biocomponent bioadhesive formulations, with a synthetic part and an autologous biological part of blood origin comprising plasma rich in platelets and in growth factors, and its use of same in biomedicine, preferably in ophthalmic surgery. In particular, he teaches a method for closing a surgical site by applying the bioadhesive.

US Patent application number: 20070244495 by Kwon titled “Apparatus and method for performing laser-assisted vascular anastomoses using biological glue” discloses methods and devices for creating vascular anastomoses are disclosed, wherein a vein is tissue welded to an artery at a desired anastomosis site. A laser is then used to vaporize tissue within the anastomosis site to form an access pathway between the vein and artery. Single-fiber or multi-fiber lasers devices may be used, and are preferably configured to emit the laser light at an angle from the longitudinal axis of the laser device to permit intravascular access to the anastomosis site. The tissue welding may be performed using a mussel or frog-derived biological glue.

Fibrin-based glue use was also documented in a number of surgical treatments. Article titled: “Optimal application of fibrin glue for a dissected aortic wall: influence of compression and compression time” by Y. Fukuhiro, describes a surgical treatment experiment of aortic dissection, where adhesives are widely used to reinforce the dissected wall. Fibrin glue has been used to facilitate hemostasis, provide suture support, and seal tissues in a variety of surgical procedures. A few reports have described the use of fibrin glue to reinforce an acutely dissected aortic wall and avoid redissection, however, the optimal method to apply this glue has not been established. The dissected aortic wall of a pig was cut into segments of 1 cm2 each and then joined with fibrin glue.

In another experimental study of effective application method of biological glue by H. Kin, T. Nakajima, H. Okabayashi from Iwate Medical University Memorial Heart Center, Morioka, Japan the most effective methods of fibrin glue as a haemostatic sealant were experimentally investigated. Three needle holes were made on the polytetrafluoroethylene graft was used. The end of it was connected to a syringe type infusion pump and the other end was connected to a monometer. The pressure was measured after leaking solution from any area of needle hole. Fibrinogen solution (A, 0.3 ml) and thrombin solution (B, 0.3 ml) of the fibrin glue was applied on the needle holes.

Another research named: “v12-28 prevention of the endoleak type ii, in the endoprostheses implantation with the injection of the thrombotic substance in the aneurysmatic sac” C. Vaquero-Puerta from Laboratory of Surgical Research and Experimental Techniques, Faculty of Medicine, Valladolid, Spain conclude that with the injection of glue in the sac should prevent the course of progressive aneurysm growth and rupture.

Another research by F. Alamanni from Department of Cardiovascular Surgery, University of Milan—Centro Cardiologico Monzino, Milan, Italy on sutureless patch and glue technique for repair of coronary sinus injuries concluded that the sutureless pericardial patch and glue technique for repair of coronary sinus injuries is safe and feasible for repair of central coronary sinus injuries.

With regards to the nanocapsules aspect of our invention, nanocapsules became known in a variety of medical applications recently. US Patent application 20040258761 by Wheatley titled “Polymer-based microcapsules and nanocapsules for diagnostic imaging and drug delivery and methods for their production” discloses methods for producing polymer-based microcapsules and nanocapsules for use in diagnostic imaging and delivery of bioactive compounds as well as targeted imaging and delivery to selected tissues and cells are provided. Compositions containing these microcapsules and nanocapsules for use in diagnostic imaging and delivery of bioactive agents are also provided. Methods for enhancing delivery of nanocapsules via ultrasound are also provided.

Nanodiamonds could provide one certain material for the nanocapsules. The unique properties of nanodiamonds make them great candidates for delivery and targeting of pharmaceutical, therapeutic, and agents for disease diagnosis, treatment, and prevention of a wide range of disease processes while minimizing side effects given their sub-cellular size. Diamond, in general, is known to be non-toxic and biocompatible which makes it a great tool for using in medical applications. Specifically, at temperatures below 500 degrees C., diamond typically does not react with other materials. Further, diamond is compatible with most biological systems, and, therefore, diamond is ideal for use in medical applications. Nanodiamonds are small particles of diamonds, typically smaller than 20 nm (generally, from about 0.5 nm to about 20 nm). In recent years, nanoparticles of diamond have become commercially available. Nanodiamond particles, with their vast number of surface atoms, can hold a large amount of such adsorbed or covalently bound atoms. Consequently, nanodiamond particles can readily attach to glue, amino acids, proteins, cells, DNA, RNA, and other biological materials. Nanodiamonds, due to its small size and spherical shape exhibit a large surface area that enhances contact with other substances and therefore the chemical reactions. There are a number of applications currently available and a number of patents/patent applications published in the field of nanodiamonds uses in biotechnology. Most of them deal with methods of drug delivery where drugs are attached to surfaces of nanodiamonds to enhance the efficacy of drugs such as chemotherapy drugs, cholesterol-reducing drugs and other substances. None of the currently available prior art documents deals with nanodiamonds used for biological glue delivery.

U.S. Pat. No. 7,294,340 by Sung titled “Healthcare and cosmetic compositions containing nanodiamonds” discloses an invention with nanodiamonds in dental and cosmetic composites.

US patent application 20100305309 by Ho titled “Nanodiamond particle complexes” describes a solution where a complex of nanodiamond particles and therapeutic agents (such as insoluble therapeutics, anthracycline and/or tetracycline compounds) is used to deliver those therapeutic agents to the designated treatment site.

Use of existing commercially available biological glue products, as well as products described in prior art cited, is problematic to use in treatment sites inside human body without an outside access. It is especially true for minimally invasive procedures, such as stent graft aorta aneurysm repair.

Use of nanodiamonds with the biological glue can be advantageous for such minimally invasive procedures, especially considering the remote release feature disclosed in our invention.

Novel feature in our invention, such as use of capsulated bi-component biological glue, as well as remote glue release in our invention opens new possibilities for use of bioglue in minimally invasive procedures.

With regards, to innovative balloon disclosed in our invention, there are a number of prior art citations. In general, medical balloon manufacturing is well known in the art. There are a few novel medical balloon manufacturing methods. U.S. Pat. No. 6,696,121 by Jung titled “Balloon for a dilation catheter and method for manufacturing a balloon” produces a the present invention relates to a balloon for a dilation catheter that is useful for performing medical dilation procedures such as angioplasty, and/or delivering a stent and a method for manufacturing the balloon.

Use of wheel-gear shaped balloon in the folded state in the disclosed invention allows for less pressure being applied when inflating the balloon and, less chances of balloon rupture while inflating.

### PRIOR ART CITATIONS

### US Patent Documents

U.S. Pat. No. 5,100,429 Sinofsky
U.S. Pat. No. 5,928,280 Hansen
U.S. Pat. No. 5,935,140 Buratto
U.S. Pat. No. 6,014,580 Blume
U.S. Pat. No. 6,019,993 Bal
U.S. Pat. No. 6,068,637 Popov
U.S. Pat. No. 6,696,121 Jung
U.S. Pat. No. 7,175,651 Kerr
U.S. Pat. No. 7,280,863 Shahar
U.S. Pat. No. 7,294,340 Sung
U.S. Pat. No. 7,348,754 Prasanna
U.S. Pat. No. 7,404,823 Gregorich
U.S. Pat. No. 7,771,442 Shriver
U.S. Pat. No. 7,851,447 Muir
U.S. Pat. No. 8,002,816 Greenberg
U.S. Pat. No. 8,043,354 Greenberg
US 20020052546—Frantz
US 20040258761 Wheatley
US 20060287713 Douglas
US 20060287714 Erbel
US 20070244495 Kwon
US 20080107305—Vanderkooy
US 20090312743 Allen
US 20090317483 Sanz
US 20100305309 Ho

### Worldwide Patent Documents

WO/2004/006795 Shahar

### Other References

Article titled: “Optimal application of fibrin glue for a dissected aortic wall: influence of compression and compression time” by Y. Fukuhiro

“v12-28 prevention of the endoleak type ii, in the endoprostheses implantation with the injection of the thrombotic substance in the aneurysmatic sac” C. Vaquero-Puerta from Laboratory of Surgical Research and Experimental Techniques, Faculty of Medicine, Valladolid, Spain

1. A method for performing minimally invasive aneurysm repair through producing a stent graft with the use of molds in a folded state, producing a gear-wheeled shape in a folded state, thus folding it to very small dimensions, delivering it to the aneurysm location, expanding it to the full expanded state, and affixing the stent to the area of aneurysm, such method comprising the steps of:
a. producing customized die for mold manufacturing;
b. mold manufacturing, such mold being produced from a wax-like material, the said mold having gear-wheel shape;
c. performing the pre-procedural imaging studies to determine individual aorta measurements for each patient;
d. trimming or otherwise tailoring the said mold according to patient individual aorta measurements;
e. producing balloon layer with the use of the mold;
f. forming a collagenous layer by dipping wax mold in the liquid collagen solution, and then melting the wax mold upon solidifying the collagen;
g. capsulation of the biological glue, and subsequent spraying of the biological glue upon the stent graft, when the biological glue is a bicomponent glue and each component is capsulated separately;
h. forming a quickly dissolvable covering layer to cover the collagenous layer and the biological glue layer;
i. connecting the abovementioned balloon with the guidewire, the said guidewire will be used as a “rail” upon which the stent graft will be implanted to the aneurysm location, such guidewire having a distal end tip attached to it, such tip providing stent graft location tracking during the implantation;
j. implanting the stent graft by guiding it to the designated aneurysm location;
k. expanding the stent graft by inflating the balloon;
l. affixing the stent graft to the designated aneurysm location by activating the biological glue at the aneurysm treatment location by using UV light means or by using ultrasound means.

2. The method of claim 1, wherein the aneurysm is ascending aorta aneurysm, descending aorta aneurysm, arch aorta aneurysm, and cerebral aneurysm.

3. The method of claim 1, wherein the biological glue is selected from a group consisting of fibrin, fibrinogen, thrombin, albumin, and myoglobin, or combination thereof.

4. The method of claim 1, wherein the biological glue is capsulated in nanocapsules.

5. The method of claim 1, wherein the gluing is performed using UV light means.

6. The method of claim 1, wherein the gluing is performed using ultrasound means.

7. Medical device for performing minimally invasive aneurysm repair, comprising:
a. a gear-wheel shaped body in the folded state made of biocompatible graft material, preferably collagenous material, and a tubular body of biocompatible graft material in its expanded state, preferably of collagenous material;
b. inside balloon-like layer, such layer having balloon-like characteristics, the said layer being folded to the shape having gear-wheel shape;
c. a guidewire, such guidewire will be connected with the balloon, the said guidewire will be a “rail” upon which the stent graft will be implanted to the aneurysm location;
d. capsulated bicomponent biological glue;
e. dissolvable layer, such layer disposed along at least a portion of the stent surface, the said layer capable of quick dissolving upon stent graft implantation.

8. Medical device of claim 7, wherein the radius of the stent graft is determined through computer simulation in such a way that it would not penetrate aorta walls, and where such radius will have the maximal possible size subject to non-penetrating condition of given closed boundaries of aorta.

9. Medical device of claim 7, wherein the said guidewire having a tip mounted at its distal end for implantation process tracking.

10. Method for biological glue preparation and activation in minimally invasive aneurysm repair proceeding, comprising the steps of:
receiving two components of the bicomponent biological glue in two separate vessels;
separately capsulating both components of the biological glue in small-sized capsules;
delivering the small-sized capsules to the medical treatment site;
activating the biological glue by using UV light means or ultrasound means.

11. Method of claim 10, wherein the capsules are nanocapsules.

12. Method of claim 10, wherein the capsules are coated with nanodiamonds.

13. Medical balloon device, such device having a gear-wheel folded shape being produced in a gear-wheel folded shape with a number of cogs changing in accordance with balloon dimensions, such balloon expanding to the inflated oval shape, wherein in such a balloon smaller pressure is needed to facilitate balloon inflation, and the probability of balloon rupture is lesser.

14. Medical balloon device of claim 13, wherein the balloon comprises polyvinyl material.

15. Method for calculating implantation path of stent graft into aorta by using computer simulation trial-and-error means, by discrete pulses by using two or more magnets through utilizing magnetic horizontal projection forces by computing the sequence of points, comprising the steps of:
determining the starting of stent graft implantation, such starting point will be in the low-left corner of the table the patient is laying on;
determining the X axis as being directed in the left-to-right direction, and the Y axis will be directed in down-upwards direction, as usual in system of coordinates, and the Z axis will be directed from the table in upwards direction;
determining stent graft weight (mass);
determining acceleration sufficient to move the stent graft for required distance D during reasonable time interval t;
defining the friction strength inside the aorta;
defining the net strength required for moving the stent graft;
defining the angle between direction from the stent graft tip to the magnet and system of coordinates plane;
defining the distance from magnet to the stent graft tip;
defining the stent moving condition in a case of two magnets (one above and one below the surgery table);
defining, through computerized simulation trial-and-error method subject to non-penetrating of aorta walls condition, the sequence of points by S₂={(Xₖ^{Q}, Yₖ^{Q}, Zₖ^{Q})}ₖ₌₁^{N}, Q=1,2; where Q is an electromagnet number (first and second), k is a number of the current point of the sequence, and N represents a number of consecutive positions of magnets required for implanting the stent graft into the treatment site location.

16. System for robotically implanting the stent graft to the aorta treatment area, comprising:
a computerized system for calculating optimal implantation path of stent graft into aorta by using computer simulation means;
stent graft implantation means;
a detector for determining the location or orientation of the stent within the body;
a housing and a drive mechanism configured to engage and to impart motion to the stent graft, wherein the drive mechanism is supported by the housing;
the guide wire support coupled to the housing allowing the stent graft to rotated with multiple degrees of freedom;
means for reading optimal implantation path and transferring them to the housing and a drive mechanism;
two or more magnets for applying electromagnetic forces for moving the stent graft, such magnets coupled to the housing and a drive mechanism.
